(19) United States
(12) Patent Application Publication
(10) Pub. No.: US 2025/0129317 A1
(43) Pub. Date: Apr. 24, 2025

(54) BIOREACTOR FOR PRODUCTION OF ORGANOIDS

(71) Applicant: UNIVERSITEIT UTRECHT HOLDING B.V., Utrecht (NL)

(72) Inventors: Kerstin SCHNEEBERGER, Utrecht (NL); Bart SPEE, Utrecht (NL); Shicheng Ye, Utrecht (NL); Gilles Sebastiaan van Tienderen, Utrecht (NL)

(73) Assignee: UNIVERSITEIT UTRECHT HOLDING B.V., Utrecht (NL)

(21) Appl. No.: 18/687,429

(22) PCT Filed: Sep. 1, 2022
(86) PCT No.: PCT/EP2022/074315

§ 371 Date: Feb. 28, 2024

(30) Foreign Application Priority Data
Sep. 1, 2021 (NL) 2029095

(51) Int. Cl. C12M 3/00; C12M 1/00; C12M 1/06; C12M 1/34; C12N 5/071

(52) U.S. Cl. CPC C12M 21/08; C12M 23/38; C12M 23/44; C12M 23/48; C12M 27/02; C12M 29/24; C12M 41/12; C12M 41/26; C12M 41/34; C12M 41/42; C12N 5/0671; C12N 2513/00; C12N 2527/00

(57) ABSTRACT

A bioreactor for the production of organoids includes at least two reactor modules for cultivation of organoids, for example allowing parallel cultivation of organoids. A method for cell culturing using the bioreactor, more specifically a method for the expansion and differentiation of organoids using the bioreactor.

The present invention relates to a bioreactor for the production of organoids comprised of at least two reactor modules for cultivation of organoids, for example allowing parallel cultivation of organoids. The present invention further relates to a method for cell culturing using the bioreactor, more specifically a method for the expansion and differentiation of organoids using the bioreactor.

With the increasing research on tissue engineering and regenerative medicine, rapid production and large amounts of cells are demanded, and bioreactors are a key requirement to meet this demand. A very recent and upcoming technique is the expansion of organoids. Organoids are “miniature organs”, produced in vitro in 3D, that resemble their organ of origin with respect to cell types and function, and can be established from a variety of organs and species. Organoids are derived from tissue stem cells, embryonic stem cells or induced pluripotent stem cells, which can self-organize in a three-dimensional culture. The production of organoids using cell culture technology, started with a shift from culturing and differentiating stem cells in 2D, to 3D culture conditions to allow for the development of the complex 3D structures of organs. As such, organoids are promising as in vitro models to study organ development and diseases. Organoids are also used to study (personalized) treatments in a laboratory, i.e. as an in vitro model which closely resembles an in vivo organ/cell of the patient, for example to test the efficacy or toxicity of future therapies.

Organoid formation is initiated by culturing and embedding stem cells or progenitor cells in a 3D environment, using a laminin-rich extracellular matrix hydrogel (mostly Matrigel or Cultrex BME). Together with a growth factor-rich medium, this facilitates proliferation and self-organization of the cells into 3D organoids. Most organoid cultures are performed using 3D static cell culture, by seeding organoids into hydrogel droplets in cell culture plates. However, this standard static organoid culture is a tedious process, requiring large amounts of materials, labor and time, such as embedding cells in droplets of Matrigel and weekly passaging of the cell culture to maintain a healthy culture. Moreover, this static culture method results in the localized accumulation of toxic waste metabolites, as well as nonhomogeneous nutrient and oxygen distribution, which results in increased heterogeneity between individual organoids resulting in suboptimal outcomes.

Next to static cell culture, spinning bioreactors or spinner flasks are promising to produce large amounts of cells rapidly, as desired with organoid cell culture and formation of 3D organoids. These bioreactors offer certain advantages over static cell culture including minimization of gradient formation (e.g., pH, nutrients, metabolites, dissolved oxygen), increased transport of oxygen and nutrients, and prevention of cell sedimentation, thus overcoming the intrinsic limitations of static culture systems. However, present bioreactors require large inoculation volumes (at least 50 mL to 200 mL) and as such also high numbers of cells, making their application for smaller experiments unsuitable and expensive and unideal for running multiple conditions in parallel. Furthermore, commercial spinner flasks require extensive incubator space due to their size and requirement for a magnetic stir plate to rotate the stir bar. In the standard size incubators that are being used in cell culture, only 6 to 7 of these bioreactors can be used for cell culture at the same time. This makes testing multiple conditions, testing cross species variations, and testing in parallel difficult.

Considering the above, there is a need in the art for a bioreactor for the rapid, efficient and cost effective production of organoids using small inoculation volumes, wherein the reactor provides improved minimization of gradient formation during cell culture, improved oxygen and nutrient transport which results in a more rapid expansion of cells and organoid production than known cell culture systems. In addition there is a need in the art for a method for the production of organoids on small and cost effective scale.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a bioreactor for the production of organoids, wherein the at least two reactor modules are comprised of a culture vessel having a volume of between 5 to 45 mL, preferably 10 to 30 mL, more preferably 15 to 25 mL, and wherein the bioreactor is comprised of at least two reactor modules for cultivation of said organoids, wherein each reactor module comprises;
at least one gas exchange portal for continues gas exchange to and from the reactor module
a stirring element inside the reactor module for providing continues mixing,
a motor in operable connection with the stirring element,
a lid or cap closing the bioreactor as a culture vessel,
   the bioreactor comprises a microcontroller that is in communication with said motor of each reactor module for controlling the speed of the stirring element per reactor module. Each reactor module includes the stirring rod in communication with an electrical engine, and a lid or cap closing the bioreactor as a culture vessel. The at least one gas exchange portal may comprise filters (e.g. 0.22 μm filters) to prevent microbial infection of the cell culture inside the reactor module.

The bioreactor of present invention provides for continues spinning in each of the modules for optimal organoid production. The reactor module is a container that holds the culture media, cells/organoid for cultivation and the stirrer element. Per reactor module the speed of spinning or mixing can be adjusted, since each reactor module comprises a stirring element and a motor that is controlled by the microcontroller of the spinning bioreactor. There is a constant movement of media and biomass, and exchange of gas in the cell culture. Each of the reactor modules of the spinning bioreactor may comprise different media and operated under different test conditions, i.e. within a single experiment or production cycle of culturing organoids or cells. This enables testing multiple conditions, and testing cross species variations. Taken the organoid-tailored size into account, it is cheaper and easier to test different conditions in parallel. Therefore, the spinning bioreactor of present invention can be used as a model or test bioreactor to optimize protocols for larger size bioreactors; such as testing of physical-chemical factors influencing cell fate (oxygen, pH, etc.), medium conditions, optimal rotational speed, etc. The conditions inside the reactor modules of the bioreactor, such as nutrient concentrations, pH, and dissolved gases (O₂, CO₂) affect the growth and functionality of the organoids. The spinning or mixing performed by the stirrer in each of the reactor modules provides a circular flow inside the reactor that facilitates homogeneous exposure of all organoids to oxygen and nutrients. Gasses are being passively exchanged via a gas exchange portal on each of the reactor modules.

Furthermore, the bioreactor of present invention is tailored to organoid expansion and differentiation. The bioreactor requires a relatively small starting volume of 5 mL (comprising for example 1×10⁴ to 1×10⁷ cells). This amounts to an at least 80% reduction of medium and cell inoculate compared to spinner flasks which require at least 30 mL starting volume, and as such facilitates the expansion and differentiation of organoids for experiments requiring fewer cells and more conditions. Compared to large volume bioreactors, it is cheaper to optimize protocols, and its smaller size makes it possible to run multiple conditions (˜64 bioreactors) in a single incubator. Furthermore, the system is micro controlled, making it very ergonomic. This small size modular spinning bioreactor comprised of multiple reactor modules greatly reduces the costs of culturing organoids.

Spinning bioreactors are promising to produce large amount of cells rapidly. Bioreactors offer certain advantages including minimization of gradient formation (e.g., pH, nutrients, metabolites, dissolved oxygen), increased transport of oxygen and nutrients, and prevention of cell sedimentation, thus overcoming the intrinsic limitations of static culture systems. As an example, we established a protocol for large-scale production of human liver organoids in commercially available spinner flasks in our group. In the spinner flasks, organoids rapidly proliferated and reached an average 40-fold cell expansion after 2 weeks, compared to 6-fold expansion in static cultures (SCs). The bioreactor of present invention is efficient for rapid production of human organoids. Result show that spinning bioreactor can achieve a 30-42-fold expansion of liver organoids in two weeks, without impairing their epithelial phenotype. While only a 10-13 fold expansion was observed in static culture under the same conditions. Further, after rapid expansion in the spinning bioreactor, organoids can be differentiated into functional hepatocyte-like cells, and the efficiency of differentiation is higher than in static culture conditions.

According to a preferred embodiment, the present invention relates to the bioreactor wherein the bioreactor is further comprised of one or more holders arranged for holding the at least two reactor modules. The holder is preferably arranged to hold multiple reactor modules and can be extended by adding more modules. By using a holder for holding multiple reactor modules the organoid cell cultures can be more easily visually observed, when placed side by side or in clusters in the holders.

According to another preferred embodiment, the present invention relates to the bioreactor, wherein the at least two reactor modules are at least 4 reactor modules, preferably at least 6 reactor modules, more preferably at least 8 reactor modules, even more preferably at least 24 reactor modules, most preferably at least 64 reactor modules.

According to another preferred embodiment, the present invention relates to the bioreactor, wherein the stirring element is a stirring rod having a length corresponding to at least 80%, more preferably at least 90% of the total length of the culture vessel of the reactor module. The stirring rod should not touch the bottom of the culture vessel. The stirring element is in operable connection to a motor in each of the reactor modules, regulating the speed of stirring in each reaction module. The stirrer has a total length that is smaller than the total length of the culture vessel of the reactor module, thereby ensuring that the stirrer element will not touch the bottom of the culture vessel. This ensures that the entire medium will be stirred, but the cells will not be “crushed”, which would happen if the stirring rod would touch the bottom of the culture vessel of the reactor module. The bioreactor of present invention does not require a magnetic plate in the bioreactor to induce spinning, motion or stirring inside the bioreactor, which simplifies the design and makes the bioreactor less expensive than commercial available bioreactors.

According to yet another preferred embodiment, the present invention relates to the bioreactor, wherein the stirring element comprises a multitude of fins or fin structures along the length of the stirring element, wherein along the length of the stirring element said fins or fin structures are separated by a space of 5 to 30 mm, preferably 8 to 20 mm, more preferably 10 to 15 mm, most preferable 5 to 15 mm. The stirring element comprises one or more fins (or wings) or fin structures that generate a flow and lifting force inside the reactor modules to ensure that the tissue culture is in constant suspension. The fins may also relate to fin structures that are along the length of the stirring element, wherein for example one fin structure is comprised of 3 to 4 fins forming one fin structure like a propeller. The stirring element in this embodiment will comprise a multitude of fin structures (forming propeller like structures) along its length. In previous bioreactors sometimes a “clotting” of cells was observed in the bioreactor, which hampers expansion of organoids. This is due to the design of the stirring rods being not optimal. We therefore designed stirring rod layouts to optimize organoid expansion and differentiation in the spinning bioreactor of present invention. Experiments have indicated that a design where all wings are fused with each other, so that no clotting can occur in the spaces between the wings provides the good results in organoid culture. Another embodiment, where the stirring element comprises a multitude of wings along the length of the rod separated by small (at least 5-15 mm) spaces between each wing or fin structure (like a comb design) along the length of the stirring element, provided an improved, more optimal environment for organoid culture having improved space to move inside the reactor module, and also provided a reduction in clotting. The space separating the fins along the length of the stirrer element should not be less that 5 mm, to prevent destruction of the organoids cells and structure in culture. The results indicate that the design with multiple wings at a distance of 5 mm from each other along the length of the stirrer element results in the most homogeneous suspension and also best organoid expansion.

According to a preferred embodiment, the present invention relates to the bioreactor, wherein the multitude of fins is at least three fins or fin structures, preferably at least five fins or fin structures, more preferably at least 7 fins or fin structures along the length of the stirring element. The fins may also relate to fin structures that are along the length of the stirring element, wherein for example one fin structure is comprised of 3 to 4 fins forming one fin structure like a propeller.

According to yet another preferred embodiment, the present invention relates to the bioreactor, wherein the at least two reactor modules are further comprised of one or more sensors selected from the group consisting of temperature, gas and pH sensor. The module further comprises a pH, temperature and gas sensor to provide information and regulate the optimal conditions for cell cultivation per reactor module.

According to another preferred embodiment, the present invention relates to the bioreactor, wherein the spinning bioreactor further comprises one or more selected from the group consisting of control panel, LCD screen, and power source.

The present invention, according to a second aspect, relates to a method for cell culturing using the bioreactor as indicated above, wherein the cell culturing is done at a cell culture volume of between 5 to 45 mL, preferably 10 to 30 mL, more preferably 15 to 25 mL. The method of present invention provides a production method of organoids on small and cost effective scale. The bioreactor can be applied for various organoids, preferably human organoids, derived from organs and tissues, such as liver, intestine, kidney, pancreas, lung, brain, spleen and heart organoid, preferably liver organoid. Also the bioreactor may be used for more conventional cell culturing that can be cultured under stirred suspension and need to be rapidly produced, such as immune cells, MSCs, EBs, antibodies, iPSCs, ESCs, and spheroids or cellular aggregates.

The present invention, according to a further aspect, relates to a method for expansion and/or differentiation of organoids, wherein the method comprises the steps of,
a) providing a bioreactor according to the present invention;
b) providing culture media and cells for the production of organoids in the two or more reactor modules;
c) culturing of the cells under culturing conditions suitable for organoid cultivation, mixing the cell culture by activating the motor of the two or more reactor modules, setting the rotational speed per reactor module between 40 to 120 rpm;
d) harvesting the organoids from the one or more reactor modules.
   According to another preferred embodiment, the present invention relates to the method for expansion and/or differentiation of organoids, wherein culturing of the cells for organoid cultivation is done at a cell culture volume of between 5 to 45 mL, preferably 10 to 30 mL, more preferably 15 to 25 mL. Preferably the culturing of the cells for organoid cultivation is done at a small total cell culture volume for production of organoids on small and cost effective scale using the bioreactor of present invention.

According to yet another preferred embodiment, the present invention relates to the method for expansion and/or differentiation of organoids, wherein the culture media and cells in step b at the start of cell cultivation have a cell culture volume of between 5 to 15 mL, preferably 6 to 12 mL, more preferably 7 to 9 mL. Due to the design of the reactor and stirrer only a small starting volume is needed to successfully expand and differentiate organoids according to the method of present invention.

According to a preferred embodiment, the present invention relates to the method for expansion and/or differentiation of organoids, wherein culturing of the cells for organoid cultivation is done for at least 10 days, preferably at least 14 days providing an average cell expansion of at least 20 fold, more preferably at least 25 fold, most preferably at least 30 fold. The method of present invention, using the bioreactor of present invention is efficient for rapid production of human organoids. Result show that the present bioreactor can achieve a 30-42-fold expansion of liver organoids in two weeks, without impairing their epithelial phenotype. While only a 10-13 fold expansion was observed in static culture under the same conditions. Further, results show that after rapid expansion in the bioreactor, organoids can be differentiated into functional hepatocyte-like cells, and the efficiency of differentiation is higher than in static culture conditions.

According to a preferred embodiment, the present invention relates to the method for expansion and/or differentiation of organoids, wherein the rotational speed in the two or more reactor modules is between 40 to 120 rpm, preferably 50 to 80 rpm, more preferably 55 to 70 rpm, most preferably 60 to 65 rpm. Results have shown that the highest fold change of cell numbers in organoid production using the bioreactor of present invention occurs at RP 60 in the case of human liver organoids, indicated that this is the most optimal speed for liver organoid expansion in the bioreactor. Organoids from different tissues have different rotation speed optima, but in general are between 40 to 120 rpm using the bioreactor of present invention.

According to another preferred embodiment, the present invention relates to the method for expansion and/or differentiation of organoids, wherein the rotational speed differs between the two or more reactor modules. Since the bioreactor is comprised of several reactor modules, the modules can each be operated under different cell culturing conditions such as stirring speed.

According to yet another preferred embodiment, the present invention relates to the method for expansion and differentiation of organoids, wherein the organoid is one or more selected from the group consisting of liver, intestine, kidney, pancreas, lung, brain, spleen and heart organoid, preferably liver organoid.

According to another preferred embodiment, the present invention relates to the method for expansion and/or differentiation of organoids, wherein the organoid is a liver organoid, and the rotational speed is between about 50 to 80 rpm, preferably about 55 to 65 rpm, more preferably about 60 rpm. Experiments show that the liver organoid expansion were the highest at 60 rpm.

According to yet another preferred embodiment, the present invention relates to the method for expansion of organoids, wherein the organoid is an intestinal organoid, and the rotational speed is between about 80 to 120 rpm, preferably about 95 to 105 rpm, more preferably about 100 rpm. For intestinal organoids, expansion rate was the highest at 100 rpm and within two weeks, intestinal organoids cultured at 100 rpm achieved a 140-fold expansion compared to approximately 40-fold expansion in static culture (SC). These results confirm that the optimal speed for intestinal organoid expansion in the bioreactor of the present invention is about 100 rpm.

The present invention will be further detailed in the following examples and figures wherein:

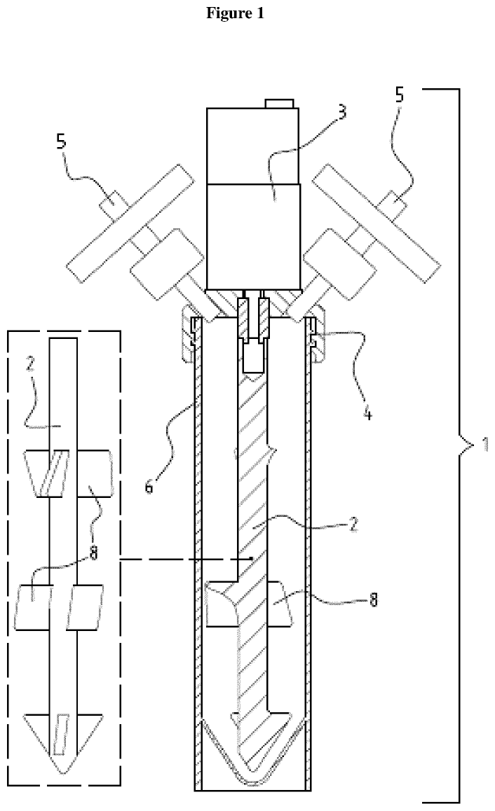

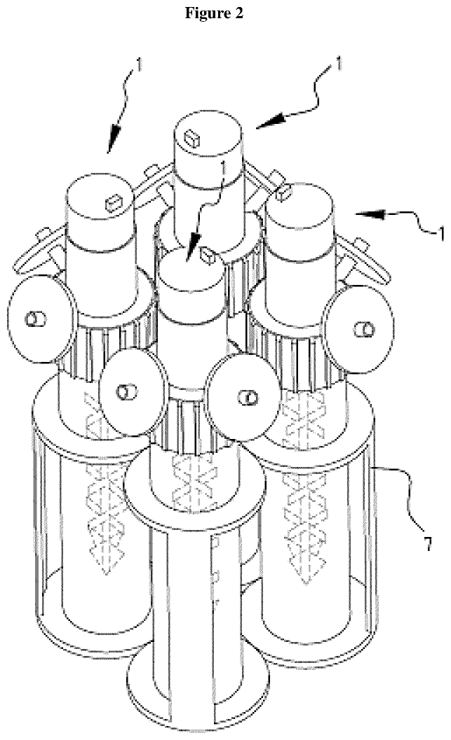

FIG. 1: Shows a schematic overview of a reactor module (1) for the production of organoids according to present invention. The reactor module includes the stirring element (2) in communication with an electrical engine (3), and a lid or cap (4) closing the reactor module as a culture vessel. The reactor module (1) further comprises portals (5) for continues gas exchange to and from the reactor module (1). The reactor module can be comprised of standard 50-mL conical tube as the culture vessel (6), for holding the culture media, cells/organoid culture and holding the stirring element (2). The stirring element (2), for example a stirring rod can be made from stainless steel and is powered by a high torque low speed electrical engine in combination with a microcontroller, for example an open-source electronic prototyping platform (Arduino) which can power individual bioreactors to operate on different rotational speeds. The stirring element (2) does not touch the bottom of the culture vessel (6) of the reactor module. The stirrer has a total length that is smaller than the total length of the culture vessel (6), thereby ensuring that the stirrer element will not touch the bottom of the culture vessel of the reactor module. This ensures that all the medium will be stirred, but the cells will not be “crushed”. The stirring element (2) comprises one or more fins (8) (or wings) that generate a flow and lifting force inside the reactor modules to ensure that the tissue culture is in constant suspension. The stirring element (2) is in operable connection to a motor (3) in each of the reactor modules (1), regulating the speed of stirring in each reaction module.

FIG. 2: Shows a holder (7) that fits up to 4 reactor modules (1) in this embodiment of present invention, that all may serve as separate bioreactors. Due to its small scale and modular build up using multiple holders, a bioreactor can for example be provided that can run at least 64 reactor modules (16 holders of 4) in a single incubator.

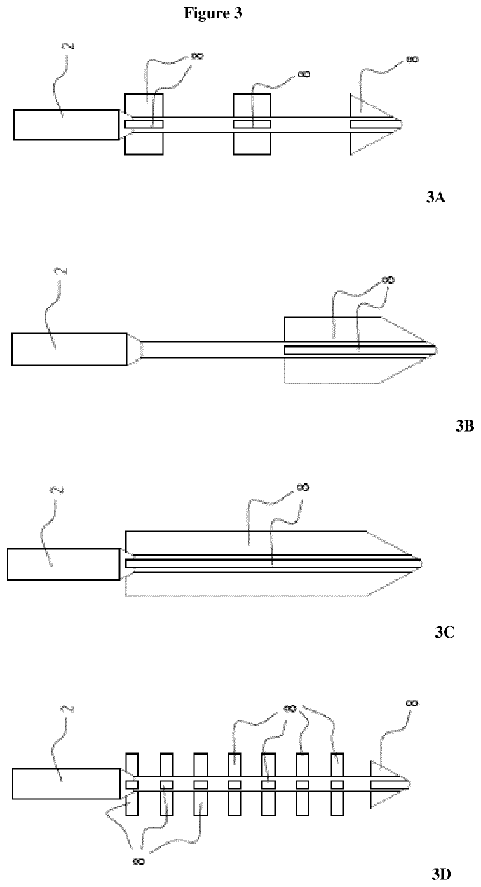

FIG. 3: Shows multiple variations (R1 to R4, FIGS. 3A to 3D respectively) on the stirring element (2) in terms of number and size of its fins (8). The stirring element (2) comprises one or more fins (8) or fin structures (or wings) that generate a flow and lifting force inside the reactor modules to ensure that the tissue culture is in constant suspension. A problem in the cultivation of organoids in bioreactors is the clotting of cells that will hamper expansion of the organoids. We therefore designed stirring rod layouts (2) to optimize organoid expansion and differentiation in the spinning bioreactor of present invention, FIG. 3A-D. Experiments have indicated that an R2 design where all wings are fused with each other forming one fin structure (FIG. 3B), so that no clotting can occur in the spaces between the wings provides good results in organoid culture. Other embodiments (R1 and R4, FIGS. 3A and 3D respectively), where the stirring element comprises a multitude of fins or fin structures along the length of the rod separated by small (at least 5-15 mm) spaces between each wing or fin structure (like a comb design wherein each fin structure forms a sort of propeller along the length of the stirrer element) along the length of the stirring element, provided an even more improved, more optimal environment for organoid culture, wherein the R4 stirrer provided the most improved results, having improved space for the cells to move inside the reactor vessel, and also provided a reduction in clotting.

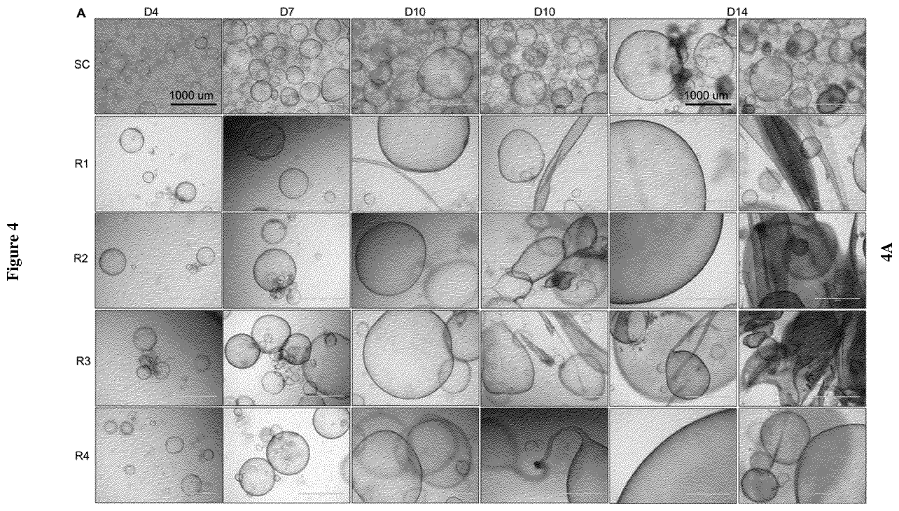

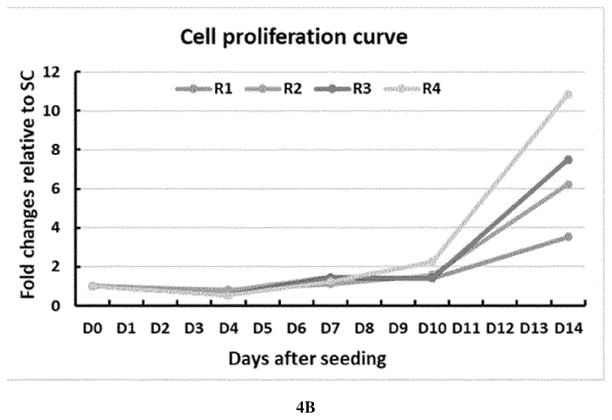

FIG. 4: Shows a comparison of organoid expansion in the bioreactor of present invention with that in static culture (SC). Furthermore the different stirring elements R1 to R4 have been tested in the bioreactor of present invention.
FIG. 4A shows the morphological track of organoids expanded in SC and in the bioreactor of present invention with different stirring rods (R1, R2, R3, R4). Bright field microscope photos were taken at four different time points (D=day. D4, D7, D10, D14) after single cell seeding. Tubular structures (right side of D10 & D14) were observed at D10. Scale bar=1,000 μm. R4 provided the most optimal organoid expansion after two weeks.
FIG. 4B shows the fold changes of cell proliferation in the bioreactor of present invention relative to static culture (SC).

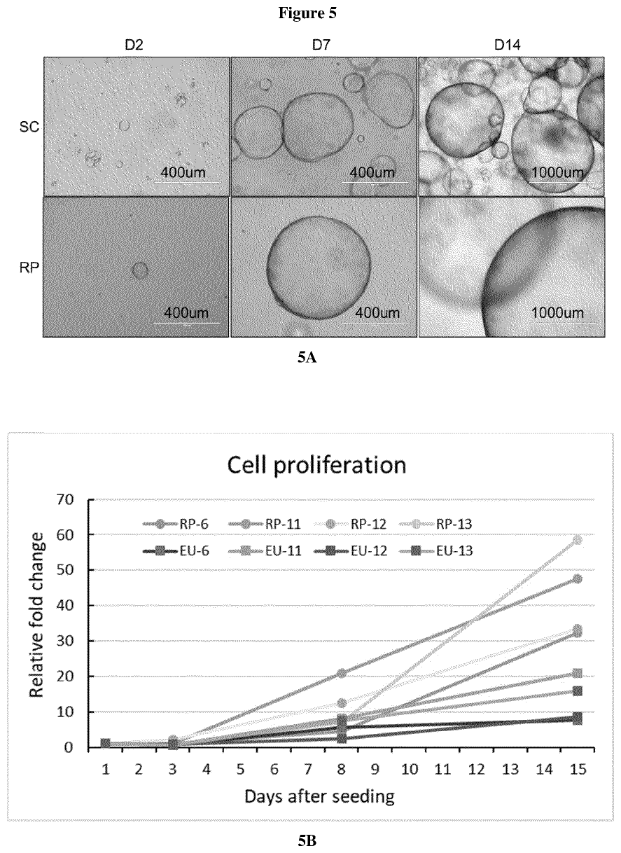

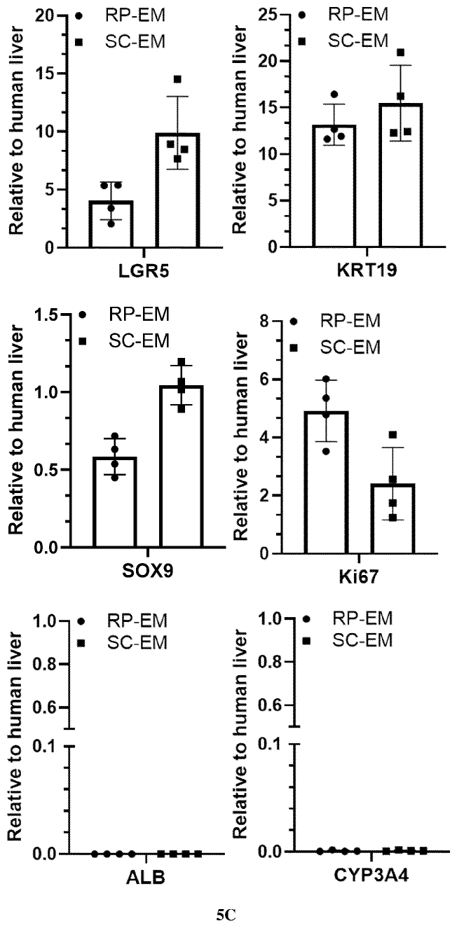

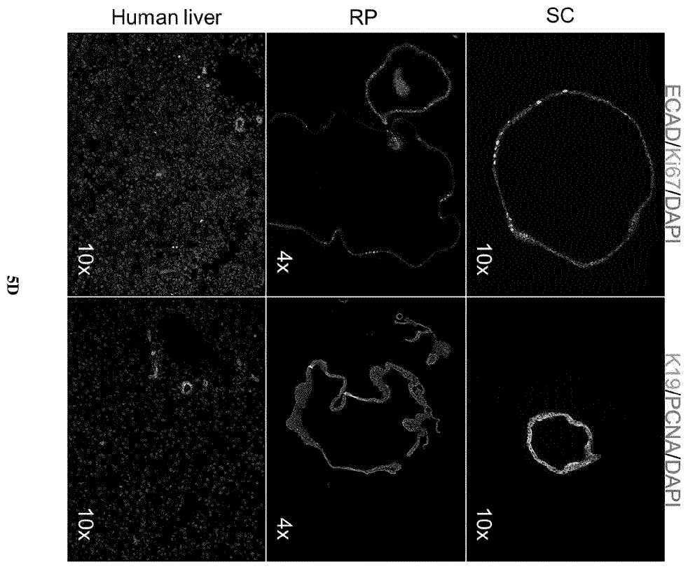

FIG. 5: Shows the expansion and characterization of human liver organoids in the bioreactor of present invention;
FIG. 5A, shows the morphology of organoids expanded in SC (static culture) and RP (bioreactor of present invention) in expansion media (EM). Pictures were taken at day 2, day 7, and day 14 after single cell seeding.
FIG. 5B, shows the growth curves of cell proliferation. A comparison of organoids expanded in static culture and in the bioreactor of present invention, indicated by fold changes relative to day 0 (D0). The numbers represent different organoid donors.
FIG. 5C, shows the mRNA expression characterized with quantitative reverse transcription polymerase chain reaction (qRT-PCR).
FIG. 5D, shows the epithelial and proliferative markers detected by immunofluorescent (IF) staining; ECAD, Ki67, DAPI, K19 and PCNA.

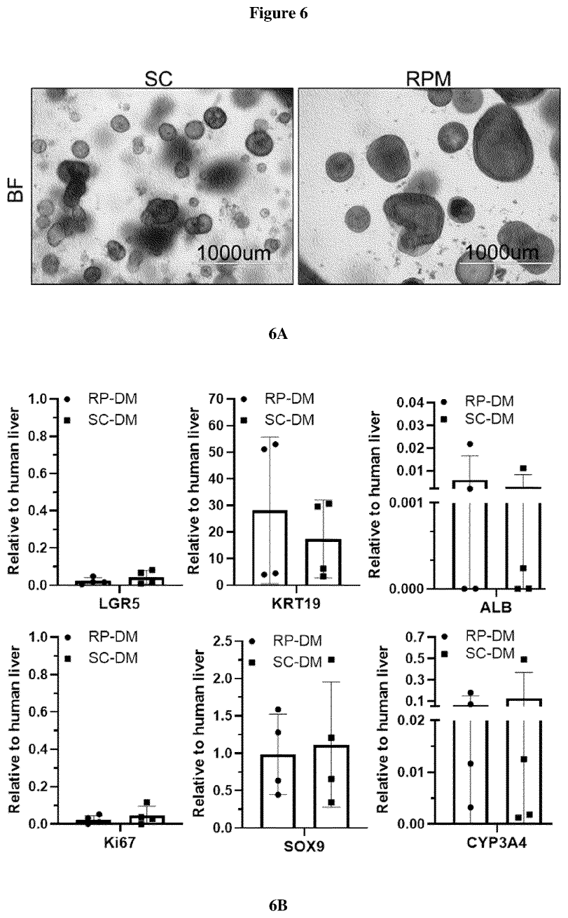

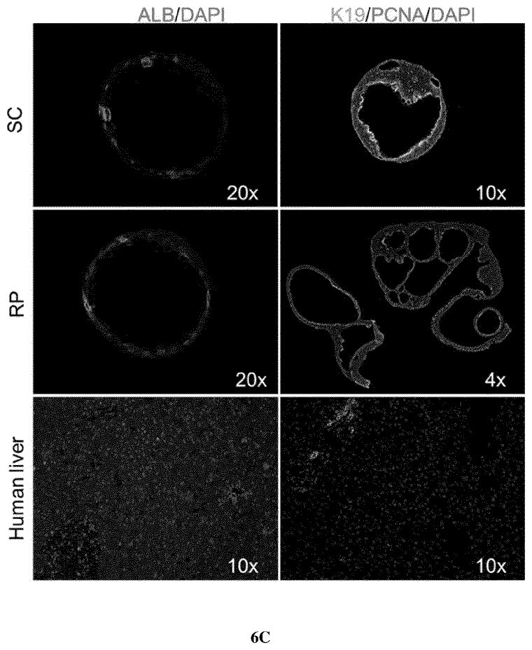

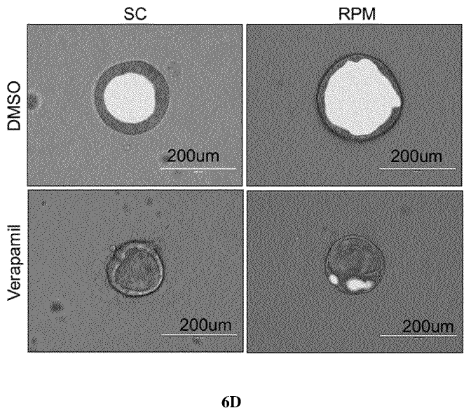

FIG. 6: Shows the characterization of human liver organoids differentiated in the bioreactor of present invention;
FIG. 6A, shows the morphology of organoids differentiated in static control (SC) and bioreactor (RP). Bright field (BF) pictures were taken after 8 days of differentiation.
FIG. 6B, shows the mRNA expression characterized with qRT-PCR.
FIG. 6C, shows the epithelial, proliferative, and functional hepatocyte markers detected by IF staining.
FIG. 6D, shows the results of the Rhodamine 123 (Rh123) transport assay.

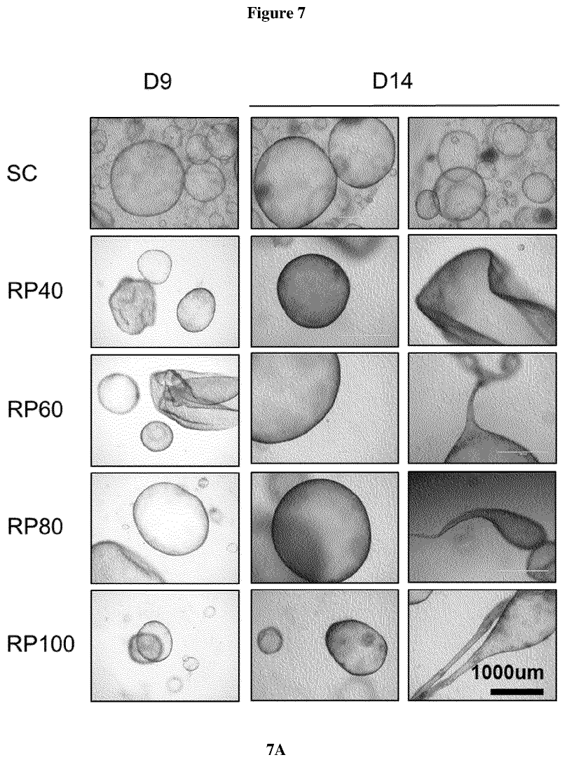

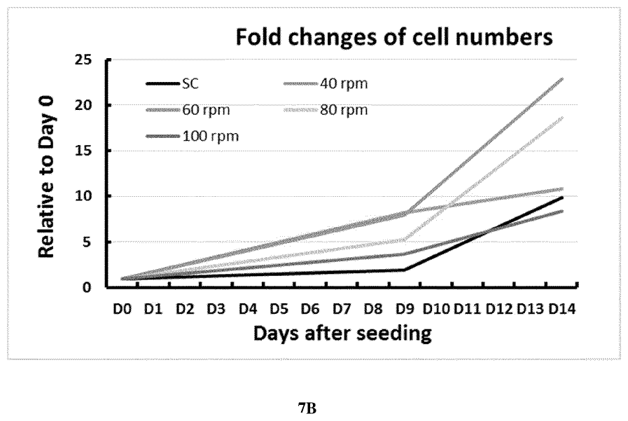

FIG. 7: Shows the optimal rotational speed for human liver organoid expansion in the bioreactor of present invention;
FIG. 7A, shows the morphology of organoids expanded in static culture and the bioreactor of present invention at four spinning speeds, 40, 60, 80, and 100 rpm. Bright field (BF) pictures were taken at day 9 and day 14 after seeding.
FIG. 7B, shows the growth curves of cell proliferation. A comparison of organoids expanded in static culture and the bioreactor at different speeds, indicated by fold changes relative to day 0 (D0).

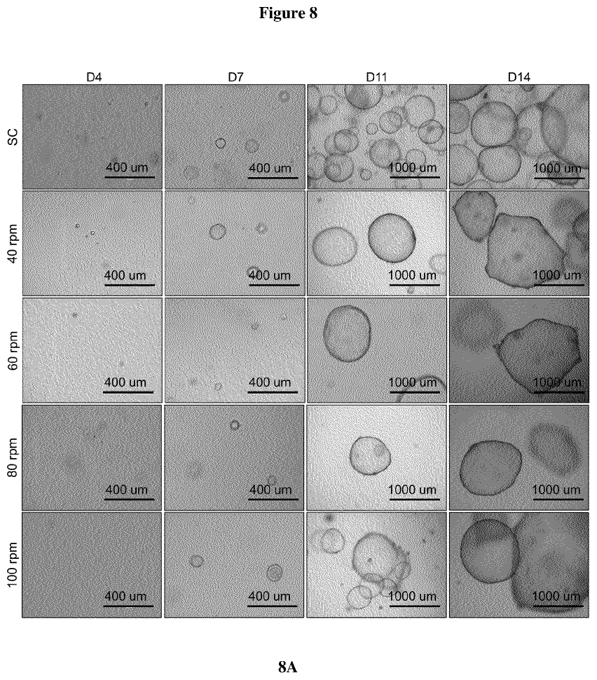

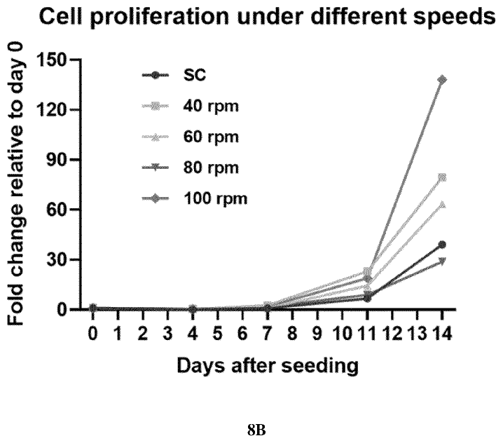

FIG. 8: Shows the optimal rotational speed for human intestinal organoid expansion in the bioreactor of present invention;
FIG. 8A, shows the morphology of organoids expanded in static culture and the bioreactor of present invention at four rotational speeds, 40, 60, 80, and 100 rpm. Bright field (BF) pictures were taken at day 4, 7, 11 and day 14 after seeding.
FIG. 8B, shows the cell proliferation curves. A comparison of organoids expanded in static culture and the bioreactor at different speeds, indicated by fold changes relative to day 0 (D0).

### EXAMPLES

### Example 1-Rapid Production and Expansion of Human Liver Organoids in the Spinning Bioreactor

To compare the expansion of organoids in the bioreactor (RP) of present invention to static cultures (SC), we seeded single cells derived from human liver organoids in both SC and RP and cultured them for two weeks in organoid expansion medium (EM). The bioreactors were inoculated with 0.5 million cells in 5 mL EM medium including 10% v/v Matrigel™. Due to single cell seeding, 10 mM Y-27632 (Rho kinase-inhibitor) was added to the medium during the first week of culture. Rotation speed was set to 80 rpm. All cultures were kept in a humified atmosphere of 95% air and 5% CO₂ at 37° C.

Every 2-3 days, new medium was added to the bioreactors. EM consisted of Advanced DMEM/F12 (Gibco, Dublin, Ireland) supplemented with 1% (v/v) penicillin-streptomycin (Gibco, Dublin, Ireland), 1% (v/v) GlutaMax (Gibco), 10 mM HEPES (Gibco), 2% (v/v) B27 supplement without vitamin A (Invitrogen, Carlsbad, CA, USA), 1% (v/v) N2 supplement (Invitrogen), 10 mM nicotinamide (Sigma-Aldrich, St Louis, MO, USA), 1.25 mM N-acetylcysteine (Sigma-Aldrich), 10% (v/v) R-spondin-1 conditioned medium (the Rspol-Fc-expressing cell line was a kind gift from Calvin J. Kuo), 10 M forskolin (FSK, Sigma-Aldrich), 5 μM A83-01 (transforming growth factor b inhibitor; Tocris Bioscience, Bristol, UK), 50 ng/mL EGF (Invitrogen, Carlsbad, CA, USA), 25 ng/mL HGF (Peprotech, Rocky Hill, NJ, USA), 0.1 μg/mL FGF10 (Peprotech) and 10 nM recombinant human (Leu15)-gastrin I (Sigma-Aldrich).

Light microscopy showed that the single cells grew out to form organoids within the first two days of culture in both SC and RP. At day 14, organoids in RP reached a diameter of up to 4 mm, compared to approximately 1 mm in SC (FIG. 5A). Cell proliferation analysis was performed at day 8 and day 15 by taking a small aliquot of cell suspension from the RP, trypsinizing organoids into single cells and subsequent single cell counting. Our results showed that in two weeks, organoids in RP achieved a 42-fold expansion on average compared to approximately 13-fold expansion in SC (FIG. 5B).

Compared to SC, organoids in RP showed a lower expression of stem cell markers (LGR5 and SOX9), but a higher expression level of the proliferation marker Ki67, indicating that a stem cell phenotype was retained in both conditions, but that in RP, the cell ratio between stem cells and highly proliferative progenitor cells was shifted towards the progenitor phenotype. Both conditions, RP and SC showed almost no expression of the functional hepatocyte markers, ALB and CYP3A4 (FIG. 5C), in line with our expectations, since organoids retain an immature and proliferative phenotype in expansion medium.

Immunofluorescent (IF) staining results confirmed their epithelial (ECAD) and highly proliferative phenotype, as indicated by a high expression of the proliferation markers Ki67 and PCNA (FIG. 5D).

Taken together, RP bioreactors are suitable for rapidly expanding liver organoids without impairing their biological liver progenitor phenotype.

Furthermore, an additional experiment was performed similar as described above, wherein various stirring elements were tested for a comparison of organoid expansion in the bioreactor of present invention with that in static culture (SC). Four different stirring elements R1 to R4 were tested in the bioreactor of present invention, wherein the stirring rods differ in design of the wings, the number of wings and the gaps between each wing section of the stirrer element (See FIG. 3 for the design differences between the R1 to R4 stirrers). FIG. 4A shows the morphological track of organoids expanded in SC and in the bioreactor of present invention with different stirring rods (R1, R2, R3, R4). Bright field microscope photos were taken at four different time points (D=day. D4, D7, D10, D14) after single cell seeding. From day 0 to day 8 no large differences were observed between the R1 and R4 design used in the bioreactor of present invention. However, from day 9 and onward, the expansion using the R4 design showed significant improvement in comparison to the other designs. Tubular structures (right pictures of FIG. 4A at D10 and D14) were observed at D10 onward. As expected no tubular formation or significant organoid expansion was observed in the SC. The R4 design in combination with the bioreactor of present invention provided the most optimal organoid expansion after two weeks. FIG. 4B show the fold changes of cell proliferation in the bioreactor of present invention relative to static culture (SC).

### Example 2-Differentiation of Human Liver Organoids in the Bioreactor

Besides organoid expansion, we also tested functional differentiation of liver organoids towards hepatocyte-like-cells (HLCs). To induce hepatic differentiation, liver organoids were primed for 2 days with the addition of 25 ng/mL BMP-7 (Peprotech, Rocky Hill, NJ, USA) to EM, after which the medium was changed to differentiation medium (DM). DM consisted of Advanced DMEM/F12 (Gibco, Dublin, Ireland) supplemented with 1% (v/v) penicillin-streptomycin (Gibco), 1% (v/v) GlutaMax (Gibco), 10 mM HEPES (Gibco), 1.25 mM N-acetylcysteine (Sigma-Aldrich, St Louis, MO, USA), 2% (v/v) B27 supplement without vitamin A (Invitrogen, Carlsbad, CA, USA), 1% (v/v) N2 supplement (Invitrogen), 50 ng/mL EGF (Invitrogen), 10 nM recombinant human (Leu15)-gastrin I (Sigma-Aldrich), 25 ng/mL HGF (Peprotech, Rocky Hill, NJ, USA), 100 ng/mL FGF19 (Peprotech), 500 nM A83-01 (Tocris Bioscience, Bristol, UK), 10 μM DAPT (Selleckchem, Munich, Germany), 25 ng/mL BMP-7 (Peprotech), and 30 μM dexamethasone (Sigma-Aldrich). Differentiation medium was changed every 2-3 days. After culture with differentiation medium (DM) for 8 days, organoids had a thick and folded morphology in both SC and RP (FIG. 6A). Gene expression analysis (mRNA analysis) by quantitative reverse transcription polymerase chain reaction (qRT-PCR) showed that the stem cell marker LGR5 and the proliferation marker Ki67 were downregulated after differentiation, while the hepatocyte markers ALB and CYP3A4 were upregulated (FIG. 6B). The ductal markers K19 and SOX9 were maintained. Gene (mRNA) expression results were verified by IF staining, particularly hepatocyte-specific protein ALB was detected in the differentiated organoids (FIG. 6C). Furthermore, Rhodamine-123 transport assays were conducted to confirm that the generated HLCs were functional. Rhodamine-123 is a fluorescent chemical compound that can be actively secreted from hepatocytes by Multidrug Resistance Gene 1 (MDR1). We observed fluorescence accumulation inside the lumen of the organoids for both SC and RP (FIG. 6D). In contrast, Rhodamine-123 was retained in the cytoplasm of the cells when organoids had been pre-treated with the competitive MDR1 inhibitor Verapamil, confirming the MDR1-specific transport of Rhodamine 123 (FIG. 6D).

To summarize, after initial rapid expansion of organoids in the bioreactor, they could subsequently be successfully differentiated into functional HLCs.

### Example 3-Optimization of the Rotation Speed for Human Liver Organoids in the Bioreactor

All initial bioreactor experiments had been performed at a rotational speeds of 80 rpm. In subsequent experiments, we continued to verify the optimal rotational speed in the bioreactor (RP). In a first experiment, four speeds, 40 rpm (RP40), 60 rpm (RP60), 80 rpm (RP80), and 100 rpm (RP100), were tested with liver organoids from one donor.

At day 9 and day 14 after seeding, representative pictures were taken, and cell numbers were counted, respectively. Bright field pictures showed that RP60 and RP80 were comparable or even better than SC at day 9. At day 14, RP60 showed the best expansion compared to all other conditions (FIG. 7A). Interestingly, some organoids appeared to be elongated as tubular structures in the RP conditions, indicating that RP conditions might be promising for better differentiation and tissue formation. The expansion was confirmed with cell counting, and the fold changes of cell numbers were consistent to the morphology, showing the highest fold change at RP60 (FIG. 7B).

### Example 4-Optimization of the Rotation Speed for Human Intestinal Organoids in the Bioreactor

A further experiment was performed to determine the optimal rotational speed for expansion of human intestinal organoids in the bioreactor (RP). Four rotational speeds, 40 rpm (RP40), 60 rpm (RP60), 80 rpm (RP80), and 100 rpm (RP100), were tested with intestinal organoids from one donor. To compare the expansion of organoids in the bioreactor (RP) at different speeds to static cultures (SC), single cells derived from human intestinal organoids were seeded in both SC and RP, and cultured for two weeks in human small intestinal organoid expansion medium.

At days 4, 7, 11 and 14 after seeding, representative pictures were taken, and cell numbers were counted, respectively. Light microscopy showed that the single cells grew out to form organoids within the first seven days of culture in both SC and RP at all rotational speeds. However, at day 14, organoids in RP reached a larger diameter compared to SC (FIG. 8A), with most and largest organoids apparent in RP100. Moreover, organoids in RP had a slightly different morphology compared to SC, with a more irregular shape at all speeds, indicating that RP conditions might be promising for better differentiation and tissue formation. The expansion was confirmed with cell counting, and the fold changes of cell numbers were consistent to the morphology, showing the highest fold change at RP100 (FIG. 8B). Within two weeks, organoids in RP100 achieved a 140-fold expansion compared to approximately 40-fold expansion in SC. These results confirm that the optimal speed for intestinal organoid expansion in the bioreactor is 100 rpm.

1. A bioreactor for the production of organoids, the bioreactor comprising:
at least two reactor modules for cultivation of said organoids, wherein the at least two reactor modules are comprised of a culture vessel having a volume of between 5 to 45 mL, and wherein each reactor module further comprises;
at least one gas exchange portal for continues gas exchange to and from the reactor module
a stirring element inside the reactor module for providing continuous mixing,
a motor in operable connection with the stirring element, and
a lid or cap closing the bioreactor as a culture vessel,
wherein the bioreactor further comprises a microcontroller in communication with said motor of each reactor module for controlling the speed of the stirring element per reactor module.

2. The bioreactor according to claim 1, wherein the bioreactor is further comprised of one or more holders arranged for holding the at least two reactor modules.

3. The bioreactor according to claim 2, wherein the holder is arranged to hold at most four reactor modules.

4. The bioreactor according to claim 1, wherein the at least two reactor modules are at least 4 reactor modules.

5. The bioreactor according to claim 1, wherein the stirring element is a stirring rod having a length corresponding to at least 80 of the total length of the culture vessel of the reactor module.

6. The bioreactor according to claim 1, wherein the stirring element comprises a multitude of fins or fin structures along the length of the stirring element, and wherein along the length of the stirring element said fins or fin structures are separated by a space of 1 to 30 mm.

7. The bioreactor according to claim 6, wherein the multitude of fins or fin structures is at least three fins or fin structures along the length of the stirring element.

8. The bioreactor according to claim 1, wherein the at least two reactor modules are further comprised of one or more sensors selected from the group consisting of temperature, gas and pH sensor.

9. The bioreactor according to claim 1, wherein the bioreactor further comprises one or more elements selected from the group consisting of control panel, LCD screen, power source.

10. A method for cell culturing using the bioreactor according to claim 1, wherein the cell culturing is done at a cell culture volume of between 5 to 45 mL.

11. The method according to claim 10, wherein the cell culturing is one or more selected from the group consisting of the cultivation of an organoid, immune cells, antibodies, stem cells, EBs, iPSCs, ESCs, and spheroids or cellular aggregates.

12. The method according to claim 11, wherein the organoids are human organoids.

13. The method according to claim 11, wherein the organoid is one or more selected from the group consisting of liver, intestine, kidney, pancreas, lung, brain, spleen and heart organoid.

14. A method for expansion and/or differentiation of organoids, the method comprising,
a) providing the bioreactor according to claim 1,
b) providing culture media and cells for the production of the organoids in the two or more reactor modules,
c) culturing of the cells under culturing conditions suitable for organoid cultivation, mixing the cell culture by activating the motor of the two or more reactor modules, setting the rotational speed per reactor module between 40 to 120 rpm, and
d) harvesting of the organoids from the one or more reactor modules.

15. The method according to claim 14, wherein the culture media and cells in step b at the start of cell cultivation have a cell culture volume of between 5 to 15 mL and/or wherein culturing of the cells for organoid cultivation is done at a cell culture volume of between 5 to 45 mL.

16. (canceled)

17. The method according to claim 14, wherein culturing of the cells for organoid cultivation is done for at least 10 days providing an average cell expansion of at least 20 fold.

18. The method according to claim 14, wherein the rotational speed in the two or more reactor modules is between 40 to 120 rpm.

19. The method according to claim 14, wherein the rotational speed differs between the two or more reactor modules.

20. The method according to claim 14, wherein the organoid is one or more selected from the group consisting of liver, intestine, kidney, pancreas, lung, brain, spleen and heart organoid.

21. The method according to claim 14, wherein the organoid is a liver organoid, and the rotational speed is between about 50 to 80 rpm; and/or wherein the organoid is an intestinal organoid and the rotational speed is between about 80 to 120 rpm.

22. (canceled)
